(19) United States
(12) Patent Application Publication
(10) Pub. No.: US 2003/0114726 A1
(43) Pub. Date: Jun. 19, 2003

(54) Novel catalytic isomerization process

(76) Inventors: Linda D. Williams, Bartlesville, OK (US); Fan-Nan Lin, Bartlesville, OK (US)

Correspondence Address: RICHMOND, HITCHCOCK,, FISH & DOLLAR, P.O. Box 2443, Bartlesville, OK, 74005, US

(21) Appl. No.: 10/025,340

(22) Filed: Dec. 19, 2001

(51) Int. Cl. C07C 5/13; C10G 35/04; C07C 5/25

(52) U.S. Cl. 585/743; 585/477; 585/665; 585/741; 585/748

(57) ABSTRACT

Deactivation of an isomerization catalyst is inhibited by charging a hydrocarbon feed having a concentration of an organic aluminum halide compound to an isomerization zone operating under isomerization conditions and containing an isomerization catalyst.

This invention relates to a novel catalytic isomerization process. In another aspect, this invention relates to a method for inhibiting the deactivation of an isomerization catalyst.

### BACKGROUND OF THE INVENTION

Catalytic isomerization is a well established refining process employed by the petroleum industry for upgrading lower-valued hydrocarbons to hydrocarbons of higher value. Typically, catalytic isomerization involves the contacting of a hydrocarbon feed with an isomerization catalyst under elevated temperatures and pressures.

Isomerization catalysts typically comprise a metal component, a halogen component, and a porous inorganic oxide support. An isomerization catalyst which has been employed widely throughout the petroleum industry comprises platinum as the metal component, chlorine as the halogen component, and alumina as the support.

In a conventional isomerization process, a reactor vessel constitutes the heart of the isomerization unit. The reactor is generally provided with a fixed bed or beds of catalyst which receive upflow or downflow feed. The reactor is provided with a heater because the reactions which take place therein are predominantly endothermic. In a typical commercial isomerization process, a hydrocarbon feed with a diluent of hydrogen or hydrogen recycled gas is charged to an isomerization reactor and contacted with the catalyst contained therein. The product is separated into a liquid fraction and vaporous effluent. The vaporous effluent, a gas rich in hydrogen, can then be used as hydrogen recycled gas in the isomerization process.

During operation of a conventional catalytic isomerization unit, the activity of the isomerization catalyst gradually declines over time. There are believed to be several causes of isomerization catalyst deactivation, including, (1) formation of coke within the pores, as well as on the surface, of the catalyst, (2) agglomeration of the catalyst metal component or components, and (3) loss of the halogen component. Deactivation of an isomerization catalyst can have the following negative impacts on the isomerization process: (1) lower isomerization percentage; (2) higher required reaction temperature; (3) higher required reaction pressure; (4) increased requirement for hydrogen; and (5) decreased selectivity.

It has recently been discovered that adding small quantities of an inorganic aluminum chloride compound to an isomerization feed during isomerization can inhibit deactivation of the isomerization catalyst. However, due to their solubility properties, inorganic aluminum chloride compounds are difficult to inject into the isomerization feed in uniform quantities.

### SUMMARY OF THE INVENTION

It is an object of the present invention to provide an improved isomerization process employing a novel method which inhibits deactivation of an isomerization catalyst.

A still further object of the present invention is to provide a method for causing the presence of an organic aluminum chloride compound in an isomerization reaction zone that overcomes the problems associated with injecting inorganic aluminum chloride compounds into an isomerization feed.

Further objects and advantages of the present invention will become apparent from consideration of the specification and appended claims.

Accordingly, one embodiment of the invention is a process comprising charging a hydrocarbon feed having a concentration of an organic aluminum halide compound to an isomerization zone operating under isomerization conditions and containing an isomenization catalyst.

Another embodiment of the invention is an isomerization process that comprises charging a hydrocarbon feed to an isomerization zone operated under isomerization conditions and introducing an organic aluminum halide compound into the hydrocarbon feed in an amount that is effective to inhibit deactivation of the isomerization catalyst.

Another embodiment of the invention is an isomerization process that comprises charging a hydrocarbon feed to an isomerization zone operated under isomerization conditions and introducing an organic aluminum halide compound and a nonmetallic chloride compound into the hydrocarbon feed both in amounts that are effective to inhibit deactivation of the isomerization catalyst.

### BRIEF DESCRIPTION OF THE DRAWING

FIG. 1 is a chart plotting the isobutane product ratio (i-C₄PR) versus time for an isomerization process employing an inorganic aluminum chloride feedstream additive at first and then employing an organic aluminum chloride feedstream additive.

### DETAILED DESCRIPTION OF THE INVENTION

The present invention is based upon the discovery that, in an isomerization process wherein a hydrocarbon feed is charged to an isomerization zone operated under isomerization conditions, the presence of an organic aluminum halide in the hydrocarbon feed can be effective to inhibit deactivation of the isomerization catalyst.

The isomerization reactor employed in practicing the present invention can be any conventional isomerization reactor known in the art. The isomerization reactor can be a stand-alone reactor or can be part of a multiple-reactor isomerization system. The isomerization reactor defines an isomerization zone which contains an isomerization catalyst, usually provided in the form of a bed of such isomerization catalyst. The catalyst bed can be fixed or moving, with fixed being the presently preferred configuration.

The isomerization catalyst can be any catalyst capable of isomerizing an isomerizable hydrocarbon. Preferably, the isomerization catalyst comprises platinum and a support material. More preferably, the isomerization catalyst comprises platinum, a halogen component, and a porous support material. Most preferably, the isomerization catalyst comprises, consists of, or consists essentially of platinum, chlorine, and an alumina support.

The hydrocarbon feed charged to the isomerization zone comprises isomerizable hydrocarbons. Any straight-chain or branched alkane containing in the range of from about 4 carbon atoms to about 10 carbon atoms per molecule can be employed as feed hydrocarbon in the isomerization process of this invention. Non-limiting examples of suitable alkanes include, but are not limited to, n-butane (presently preferred), n-pentane, n-hexane (also presently preferred), 2-methylpentane, 3-methylpentane, n-heptane, 2-methylhexane, 3-methylhexane, octanes, nonanes, decanes, and the like and mixtures thereof.

Any cycloalkane containing in the range of from about 5 carbon atoms to about 10 carbon atoms per molecule can also be used as feed hydrocarbon in the process of this invention. Non-limiting examples of suitable cycloalkanes include, but are not limited to, cyclopentane, cyclohexane, methylcyclopentane, cycloheptane, methylcyclohexane, cyclooctane, methylcyclooctane, and the like and mixtures thereof.

Mixtures of alkanes and cycloalkanes, in any proportion, such as a molar ratio of alkane to cycloalkane of from about 1:99 to about 99:1, can also be employed as feed hydrocarbon in the isomerization process of this invention.

A diluent can be added to the hydrocarbon feed prior to introducing the hydrocarbon feed into the isomerization reactor. Any diluent recognized in the art can be utilized either individually or in admixture with hydrogen. Hydrogen is the presently preferred diluent because it serves the dual function of lowering the partial pressure of the hydrocarbon feed and suppressing the formation of coke on the isomerization catalyst. The weight ratio of diluent-to-hydrocarbon feed is preferably maintained at from about 0.01:1 to about 20:1, more preferably from about 0.02:1 to about 5:1, and most preferably from 0.05:1 to 3:1.

In the process of this invention, impurities can also be present in the isomerization feed stream. These impurities can include, but are not limited to, sulfur compounds, water, carbon dioxide, carbon monoxide, aromatic hydrocarbons containing in the range of from about 6 carbon atoms to about 10 carbon atoms per molecule, such as, for example, benzene, toluene, and xylene, olefin hydrocarbons containing in the range of from about 2 carbon atoms to about 10 carbon atoms per molecule, and the like and combinations thereof. The amounts of these additional impurities should be small enough that the impurities do not have detrimental effects on the process of this invention. Generally, the total content of these impurities, if present, in the isomerization feed stream (on an elemental basis, based on the total weight of the hydrocarbon feed) is in the range of from about 1 ppm impurity to about 2,000 ppm impurity (i.e., about 1 to about 2,000 parts by weight of impurity per million parts by weight of the hydrocarbon feed). In most cases, the impurity content is in the range of from about 10 ppm to about 200 ppm.

The temperature required for isomerization varies according to numerous reaction parameters, including, for example, feed composition, catalyst composition, pressure, amount of diluent, and the amount of coke on the isomerization catalyst. Generally, the temperature required for isomerization is in the range of from about 100° F. to about 600° F., more preferably from about 120° F. to about 5750° F., and most preferably from 140° F. to 550° F. Ordinarily, the temperature is slowly increased during the isomerization process to compensate for deactivation of the catalyst.

The isomerization reaction pressures are in the range of from about 150 psig to about 1500 psig, preferably from about 200 psig to about 1200 psig, and most preferably from 250 psig to 1000 psig.

The liquid-volume hourly space velocity (LHSV) of the hydrocarbon feed to the isomerization zone is in the range of from exceeding 0 to about 1000 hours⁻¹. The preferred LHSV of the hydrocarbon feed can be in the range of from about 0.25 to about 250 hours⁻¹.

In accordance with an embodiment of the present invention, it is essential for an organic aluminum halide compound to be injected into the hydrocarbon feed, at least for a time period, during isomerization. It has been discovered that organic aluminum halide compounds are superior isomerization feed additives to inorganic aluminum chloride compounds because, for example, organic aluminum halide compounds are more soluble in hydrocarbon feeds than inorganic aluminum chloride compounds. The solubility properties of organic aluminum halide compounds allow for reliable, uniform injection into the hydrocarbon feed without clogging the injection means.

Any organic aluminum halide compound that is effective to inhibit deactivation of an isomerization catalyst can be used in practicing the process of the present invention. Preferably, the organic aluminum halide compound is represented by the formula R_{3-y}AlX_{y} wherein R is an alkyl, alkenyl, or aryl radical containing 1 to 6 carbon atoms, X is a halogen, and Y is from 1 to 2. Preferably, R is an alkyl radical containing 1 to 4 carbon atoms and X is a halogen selected from the group consisting of chlorine and bromine. Most preferably, R is ethyl, X is chlorine, and Y is 2.

Preferred organic aluminum halide compounds include, for example, dimethyl aluminum chloride, diethyl aluminum chloride, di-n-propyl aluminum chloride, di-isobutyl aluminum chloride, di-n-butyl aluminum chloride, methyl aluminum sesqui-chloride, ethyl aluminum sesqui-chloride, n-propyl aluminum sesqui-chloride, isobutyl aluminum sesqui-chloride, n-butyl aluminum sesqui-chloride, methyl aluminum dichloride, ethyl aluminum dichloride, n-propyl aluminum dichloride, isobutyl aluminum dichloride, n-butyl aluminum dichloride, dimethyl aluminum bromide, diethyl aluminum bromide, di-n-propyl aluminum bromide, di-isobutyl aluminum bromide, di-n-butyl aluminum bromide, methyl aluminum sesqui-bromide, ethyl aluminum sesqui-bromide, n-propyl aluminum sesqui-bromide, isobutyl aluminum sesqui-bromide, n-butyl aluminum sesqui-bromide, methyl aluminum dibromide, ethyl aluminum dibromide, n-propyl aluminum dibromide, isobutyl aluminum dibromide, n-butyl aluminum dibromide, and combinations or two of more thereof. More preferred organic aluminum halide compounds include, for example, dimethyl aluminum chloride, diethyl aluminum chloride, methyl aluminum dichloride, ethyl aluminum dichloride, dimethyl aluminum bromide, diethyl aluminum bromide, methyl aluminum dibromide, ethyl aluminum dibromide, and combinations of any two or more thereof. The presently most preferred organic aluminum halide compound is ethyl aluminum dichloride.

The organic aluminum halide compound can be injected into the hydrocarbon feed at a point located immediately upstream from the inlet of the isomerization reactor. As used herein, the phrase “immediately upstream from the inlet of the isomerization reactor” means a location wherein there is no substantial change in the composition of the hydrocarbon feed and the additive between the additive injection point and the inlet of the isomerization reactor.

The organic aluminum halide compound can be injected in pure form or with a carrier. Preferably, the organic aluminum halide compound is injected with a carrier. The carrier can be any compound capable of dissolving the organic aluminum halide compound which does not have an adverse material impact on the isomerization reaction. Preferably, the carrier is a hydrocarbon.

The organic aluminum halide compound can be injected into the hydrocarbon feed by any method known in the art. It is preferred for the organic aluminum halide compound to be injected immediately upstream from the reactor.

A preferred injection system comprises an additive storage source connected in fluid flow communication with an additive moving means connected in fluid flow communication with an additive flow control means connected in fluid flow communication with an additive injection means. The additive storage source can be any conventional means of storing a quantity of a compound such as an organic aluminum halide compound, for example, a storage tank. The additive moving means can be any conventional means of moving a quantity of a compound such as an organic aluminum halide compound through a conduit, for example, a pump. The additive flow control means can be any conventional means for controlling the flow of a compound such as an organic aluminum halide compound to an isomerization reactor, for example, a valve or valves. The additive injection means can be any conventional means for injecting a compound such as an organic aluminum halide compound into a conduit carrying a hydrocarbon feed, for example, a nozzle or quill.

The rate of injection of the organic aluminum halide compound into the hydrocarbon feed can be any rate suitable for inhibiting deactivation of the isomerization catalyst. Preferably, the injection rate is sufficient to provide a concentration of the organic aluminum halide compound in the hydrocarbon feed of from about 0.001 ppbw to about 500 ppbw. More preferably, the injection rate provides a concentration of the organic aluminum halide compound in the hydrocarbon feed of from about 0.01 ppbw to about 100 ppbw. Still more preferably, the injection rate provides a concentration of the organic aluminum halide compound in the hydrocarbon feed of from about 0.1 ppbw to about 50 ppbw. Most preferably, the injection rate provides a concentration of the organic aluminum halide compound in the hydrocarbon feed of from 0.5 ppbw to 15 ppbw.

The period of organic aluminum halide compound injection into the hydrocarbon feed can be any suitable period that is effective to inhibit deactivation of the isomerization catalyst. The organic aluminum halide compound can be injected continuously or intermittently, with intermittent injection being preferred. The intermittent injection method can comprise an injection period of from about 0.1 hours to about 100 hours, and a noninjection period which is from about 1 to about 20 times longer than the injection period. Preferably, the injection period is from 1.0 to 50 hours and the noninjection period is from 2 to 5 times longer than the injection period.

In accordance with an embodiment of the present invention, in addition to the presence of an organic aluminum halide compound in the hydrocarbon feed, a nonmetallic chloride compound can also be present in the hydrocarbon feed during isomerization.

The nonmetallic chloride compound can be any nonmetallic chloride-containing compound suitable for inhibiting the deactivation of an isomerization catalyst when used in combination with an organic aluminum halide compound and capable of reacting with an organic aluminum halide compound under isomerization conditions to produce an inorganic aluminum chloride compound. Preferably, the nonmetallic chloride compound is selected from the group consisting of perchloroethylene, hexachloroethane, carbon tetrachloride, 1-chlorobutene, 1-chloro-2-methylpropane, 2-chloro-2-methylpropane propylene dichloride, hydrogen chloride, and mixtures of any two or more thereof. More preferably, the nonmetallic chloride compound is selected from the group consisting of perchloroethylene, hydrogen chloride, and mixtures thereof. Most preferably, the nonmetallic chloride compound is perchloroethylene.

The amount of nonmetallic chloride compound injected into the isomerization feed can be any amount which, when used in combination with an organic aluminum halide compound is suitable for inhibiting the deactivation of the isomerization catalyst and reacting with organic aluminum halide compounds under isomerization conditions to produce inorganic aluminum chloride compounds in the isomerization reactor. Preferably, the amount of nonmetallic chloride compound injected is an amount which is sufficient to provide a concentration of the nonmetallic chloride compound in the hydrocarbon feed in the range of from about 0.01 ppmw to about 500 ppmw, more preferably from about 0.1 ppmw to about 100 ppmw, still more preferably from about 0.2 ppmw to about 50 ppmw, and most preferably from 0.5 ppmw to 10 ppmw.

The weight ratio of organic aluminum halide compound to nonmetallic chloride compound in the hydrocarbon feed can be from about 1:100,000 to about 1:10, preferably from about 1:10,000 to about 1:50, and most preferably from 1:5,000 to 1:500.

The organic aluminum halide compound and the nonmetallic chloride compound can be injected into the hydrocarbon feed at separate injection points, or can be mixed prior to injection and introduced into the hydrocarbon feed together. The nonmetallic chloride compound is preferably injected immediately upstream from the inlet of the isomerization reactor. The nonmetallic chloride compound can be injected into the hydrocarbon feed by any method known in the art, including in the same manner described above for injecting the organic aluminum halide compound.

The nonmetallic chloride compound can be injected in pure form or with a carrier. Preferably, the nonmetallic chloride compound is injected with a carrier. The carrier can be any compound capable of dissolving the nonmetallic chloride compound which does not have an adverse material impact on the isomerization reaction. Preferably, the carrier is a hydrocarbon.

The period of injection for the nonmetallic chloride compound in combination with the organic aluminum halide compound can be any suitable period that is effective to inhibit deactivation of the isomerization catalyst. The nonmetallic chloride compound can be injected continuously or intermittently, with intermittent injection being preferred. The intermittent injection method can comprise an injection period of from about 0.1 hours to about 100 hours, and a non-injection period which is from about 1.0 to about 20 times longer than the injection period. Preferably, the injection period is from 10 to 50 hours and the non-injection period is from 2 to 5 times longer than the injection period.

### EXAMPLE I

The following example is presented to further illustrate the invention and is not to be considered as limiting the scope of the invention.

This example demonstrates that an organic aluminum chloride feedstream additive is at least as effective as an inorganic aluminum chloride feedstream additive for maintaining and/or promoting the activity of an isomerization catalyst.

A stainless-steel reactor having an inside diameter of about 0.75 inches and a height of about 28 inches was filled with a bottom layer of about 14.75 inches of inert alumina particles having a surface area of 1 m²/g or less, a middle layer of about 5.75 inches of I-8 platinum/chlorided alumina isomerization catalyst (marketed by UOP, Des Plaines, Ill.) and a top layer of about 7 inches of inert alumina particles having a surface area of 1 m²/g or less.

The reactor was brought to reaction conditions. The reaction conditions employed during the experimental run of this example included a reaction temperature of about 280° F. and a reaction pressure of about 450 psig.

A liquid hydrocarbon feed was charged to the reactor. The hydrocarbon feed contained about 98 liquid-volume % n-butane. The liquid-volume hourly space velocity of the naphtha feed was about 4 hr⁻¹. Hydrogen was charged to the reactor simultaneously with the hydrocarbon feed. The hydrogen to hydrocarbon (H₂:HC) ratio of the charge to the reactor was 0.28.

Perchloroethylene (PCE) was added to the hydrocarbon feed prior to entering the reactor. In addition, during separate periods of the experimental run of this example, an inorganic aluminum chloride compound (AlCl₃) and an organic aluminum chloride compound (EtAlCl₂) were added to the hydrocarbon feed. Table I shows the amount of PCE, AlCl₃, and EtAlCl₂ added to the hydrocarbon feed.

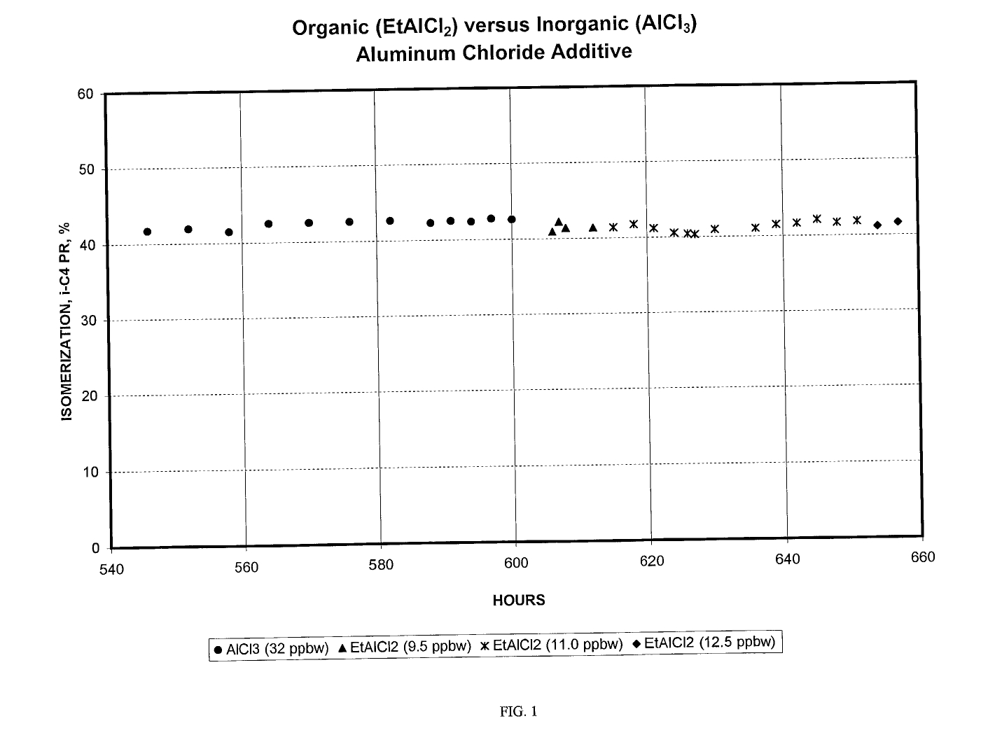

The Isobutane Product Ratio (i-C₄PR) was measured. The term “Isobutane Product Ratio”, as used herein, means: the weight percent isobutane in the product divided by the total weigh percent of isobutane and n-butane in the product. Table I provides the measured i-C₄PR values. FIG. 1 plots i-C₄PR as a function of time for the experimental run of the present example.

As illustrated in Table 1 and FIG. 1, the organic aluminum chloride compound is at least as effective as the inorganic aluminum chloride compound for maintaining and/or promoting the activity of the isomerization catalyst.

TABLE I

| Time | H₂:HC | PCE | AlCl₃ | EtA1Cl₂ | |
|---|---|---|---|---|---|
| (hours) | (wt. ratio) | (ppmw) | (ppbw) | (ppbw) | i-C₄PR |
| 546 | 0.28 | 208 | 32 | — | 41.72 |
| 552 | 0.28 | 208 | 32 | — | 41.92 |
| 558 | 0.28 | 208 | 32 | — | 41.48 |
| 564 | 0.28 | 208 | 32 | — | 42.51 |
| 570 | 0.28 | 208 | 32 | — | 42.53 |
| 576 | 0.28 | 208 | 32 | — | 42.58 |
| 582 | 0.28 | 208 | 32 | — | 42.63 |
| 588 | 0.28 | 208 | 32 | — | 42.28 |
| 591 | 0.28 | 208 | 32 | — | 42.51 |
| 594 | 0.28 | 208 | 32 | — | 42.35 |
| 597 | 0.28 | 208 | 32 | — | 42.74 |
| 600 | 0.28 | 208 | 32 | — | 42.58 |
| 606 | 0.28 | 93.5 | — | 9.5 | 40.92 |
| 607 | 0.28 | 93.5 | — | 9.5 | 42.19 |
| 608 | 0.28 | 93.5 | — | 9.5 | 41.37 |
| 612 | 0.28 | 93.5 | — | 9.5 | 41.38 |
| 615 | 0.28 | 109 | — | 11.0 | 41.37 |
| 618 | 0.28 | 109 | — | 11.0 | 41.76 |
| 621 | 0.28 | 109 | — | 11.0 | 41.19 |
| 624 | 0.28 | 109 | — | 11.0 | 40.55 |
| 626 | 0.28 | 109 | — | 11.0 | 40.40 |
| 627 | 0.28 | 109 | — | 11.0 | 40.33 |
| 630 | 0.28 | 109 | — | 11.0 | 40.97 |
| 636 | 0.28 | 109 | — | 11.0 | 41.04 |
| 639 | 0.28 | 109 | — | 11.0 | 41.53 |
| 642 | 0.28 | 109 | — | 11.0 | 41.66 |
| 645 | 0.28 | 109 | — | 11.0 | 42.14 |
| 648 | 0.28 | 109 | — | 11.0 | 41.70 |
| 651 | 0.28 | 109 | — | 11.0 | 41.82 |
| 654 | 0.28 | 125 | — | 12.5 | 41.16 |
| 657 | 0.28 | 125 | — | 12.5 | 41.62 |
| 660 | 0.28 | 125 | — | 12.5 | 40.42 |

While this invention has been described in detail for the purpose of it should not be construed as limited thereby but intended to cover all changes cations within the spirit and scope thereof.

That which is claimed is:

1. A process comprising charging a hydrocarbon feed having a concentration of at least one organic aluminum halide compound to an isomerization zone operating under isomerization conditions and containing an isomerization catalyst.

2. A process according to claim 1 wherein said organic aluminum halide compound is represented by the formula R_{3-y}AlX_{y} wherein R is an alkyl, alkenyl, or aryl radical containing 1 to 6 carbon atoms, X is a halogen, and Y is an integer selected from the group consisting of 1 and 2.

3. A process according to claim 1 wherein said concentration of said organic aluminum halide compound is in the range of from about 0.001 ppbw to about 500 ppbw.

4. A process according to claim 1 wherein said concentration of said organic aluminum halide compound is in the range of from about 0.01 ppbw to about 100 ppbw.

5. A process according to claim 1 wherein said concentration of said organic aluminum halide compound is in the range of from about 0.1 ppbw to about 50 ppbw.

6. A process according to claim 1 wherein said concentration of said organic aluminum halide compound is in the range of from 0.5 ppbw to 15 ppbw.

7. A process according to claim 1 wherein said isomerization catalyst comprises platinum, chlorine, and alumina.

8. A process according to claim 1 wherein said organic aluminum halide compound is represented by the formula R_{3-y}AlX_{y}, wherein R is an alkyl radical containing 1 to 4 carbon atoms, X is a halogen selected from the group consisting of chlorine and bromine, and Y is an integer selected from the group consisting of 1 and 2.

9. A process according to claim 1 wherein said organic aluminum halide compound is ethyl aluminum dichloride.

10. An isomerization process comprising the steps of:
charging a hydrocarbon feed comprising an isomerizable hydrocarbon to an isomerization zone containing an isomerization catalyst, wherein said isomerization zone is operated under isomerization conditions; and
introducing an organic aluminum halide compound into said hydrocarbon feed in an amount that is effective to inhibit deactivation of said isomerization catalyst.

11. A process according to claim 10 wherein said organic aluminum halide compound is represented by the formula R_{3-y}AlX_{y}, wherein R is an alkyl, alkenyl, or aryl radical containing 1 to 6 carbon atoms, X is a halogen, and Y an integer selected from the group consisting of 1 and 2.

12. A process according to claim 10 wherein said amount of said organic aluminum halide compound introduced into said hydrocarbon feed is such as to provide a concentration of said organic aluminum halide in said hydrocarbon feed of from about 0.001 ppbw to about 500 ppbw.

13. A process according to claim 10 wherein said amount of said organic aluminum halide compound introduced into said hydrocarbon feed is such as to provide a concentration of said organic aluminum halide in said hydrocarbon feed of from about 0.01 ppbw to about 100 ppbw.

14. A process according to claim 10 wherein said amount of said organic aluminum halide compound introduced into said hydrocarbon feed is such as to provide a concentration of said organic aluminum halide in said hydrocarbon feed of from about 0.1 ppbw to about 50 ppbw.

15. A process according to claim 10 wherein said amount of said organic aluminum halide compound introduced into said hydrocarbon feed is such as to provide a concentration of said organic aluminum halide in said hydrocarbon feed of from 0.5 ppbw to 15 ppbw.

16. A process according to claim 10 wherein said isomerization catalyst comprises platinum, chlorine, and alumina.

17. A process according to claim 10 wherein said organic aluminum halide compound is represented by the formula R_{3-y}AlX_{y}, wherein R is an alkyl radical containing 1 to 4 carbon atoms, X is a halogen selected from the group consisting of chlorine and bromine and Y is an integer selected from the group consisting of 1 and 2.

18. A process according to claim 10 wherein said organic aluminum halide compound is ethyl aluminum dichloride.

19. An isomerization process comprising the steps of:
charging a hydrocarbon feed comprising an isomerizable hydrocarbon to an isomerization zone containing an isomerization catalyst, wherein said isomerization zone is operated under isomerization conditions; and
introducing a first amount of an organic aluminum halide compound and a second amount of a nonmetallic chloride compound into said hydrocarbon feed, wherein said first amount and said second amount are effective to inhibit deactivation of said isomerization catalyst.

20. A process according to claim 19 wherein said organic aluminum halide compound is represented by the formula R_{3-y}AlX_{y}, wherein R is an alkyl, alkenyl, or aryl radical containing 1 to 16 carbon atoms, X is a halogen, and Y is an integer selected from the group consisting of 1 and 2.

21. A process according to claim 19 wherein said nonmetallic chloride compound is selected from the group consisting of perchloroethylene, tetrachloroethylene, hexachloroethane, carbon tetrachloride, 1-chlorobutene, 1-chloro-2-methylpropane, 2-chloro-2-methylpropane, propylene dichloride, hydrogen chloride, and mixtures of any two or more thereof.

22. A process according to claim 19 wherein said first amount of said organic aluminum halide compound introduced into said hydrocarbon feed is such as to provide a concentration of said organic aluminum halide in said hydrocarbon feed of from about 0.001 ppbw to about 500 ppbw, and wherein said second amount of said nonmetallic chloride compound introduced into said hydrocarbon feed is such as to provide a concentration of said nonmetallic chloride compound in said hydrocarbon feed of from about 0.01 ppmw to about 500 ppmw.

23. A process according to claim 19 wherein said first amount of said organic aluminum halide compound introduced into said hydrocarbon feed is such as to provide a concentration of said organic aluminum halide in said hydrocarbon feed of from about 0.01 ppbw to about 100 ppbw, and wherein said second amount of said nonmetallic chloride compound introduced into said hydrocarbon feed is such as to provide a concentration of said nonmetallic chloride compound in said hydrocarbon feed of from about 0.01 ppmw to about 100 ppmw.

24. A process according to claim 19 wherein said first amount of said organic aluminum halide compound introduced into said hydrocarbon feed is such as to provide a concentration of said organic aluminum halide in said hydrocarbon feed of from about 0.1 ppbw to about 50 ppbw, and wherein said second amount of said nonmetallic chloride compound introduced into said hydrocarbon feed is such as to provide a concentration of said nonmetallic chloride compound in said hydrocarbon feed of from about 0.2 ppmw to about 50 ppmw.

25. A process according to claim 19 wherein said first amount of said organic aluminum halide compound introduced into said hydrocarbon feed is such as to provide a concentration of said organic aluminum halide in said hydrocarbon feed of from 0.5 ppbw to 15 ppbw, and wherein said second amount of said nonmetallic chloride compound introduced into said hydrocarbon feed is such as to provide a concentration of said nonmetallic chloride compound in said hydrocarbon feed of from 0.5 ppmw to 10 ppmw.

26. A process according to claim 19 wherein the weight ratio of said organic aluminum halide compound to said nonmetallic chloride compound in said hydrocarbon feed is from about 1:100,000 to about 1:10.

27. A process according to claim 19 wherein the weight ratio of said organic aluminum halide compound to said nonmetallic chloride compound in said hydrocarbon feed is from about 1:10,000 to about 1:50.

28. A process according to claim 19 wherein the weight ratio of said organic aluminum halide compound to said nonmetallic chloride compound in said hydrocarbon feed is from about 1:5,000 to 1:500.

29. A process according to claim 19 wherein said isomerization catalyst comprises platinum, chlorine and alumina.

30. A process according to claim 19 wherein said organic aluminum halide compound is represented by the formula R_{3-y}AlX_{y}, wherein R is an alkyl radical containing 1 to 4 carbon atoms, X is a halogen selected from the group consisting of chlorine and bromine, and Y is an integer selected from the group consisting of 1 and 2.

31. A process according to claim 19 wherein said organic aluminum halide compound is ethyl aluminum dichloride.

32. A process according to claim 19 wherein said nonmetallic chloride compound is perchloroethylene.
